(19) United States
(12) Patent Application Publication
(10) Pub. No.: US 2022/0096507 A1
(43) Pub. Date: Mar. 31, 2022

(54) Parenteral Pharmaceutical Composition Comprising Azacitidine or Decitabine in DMSO

(71) Applicant: Sandoz AG, Basel (CH)

(72) Inventors: Arpad Toldy, Kundl (AT); Eva Littringer, Kundl (AT); Katri Rouvali Oberer, Kundl (AT)

(73) Assignee: Sandoz AG, Basel (CH)

(21) Appl. No.: 17/423,210

(22) PCT Filed: Jun. 29, 2021
(86) PCT No.: PCT/EP2021/067796

§ 371 Date: Dec. 20, 2021

(30) Foreign Application Priority Data
Jul. 1, 2020 (EP) 20183501.4

(51) Int. Cl. A61K 31/706; A61K 47/20; A61K 9/00; A61K 41/17; A61K 9/08

(52) U.S. Cl. CPC A61K 31/706; A61K 47/20; A61K 9/08; A61K 41/17; A61K 9/0019

(57) ABSTRACT

The invention relates to a parenteral pharmaceutical composition comprising azacitidine and dimethyl sulfoxide.

### FIELD OF THE INVENTION

The present invention is directed to a parenteral pharmaceutical composition comprising azacitidine and dimethyl sulfoxide or decitabine and dimethyl sulfoxide.

### BACKGROUND OF THE INVENTION

Azacitidine (IUPAC name 5-azacytidine) is a cytotoxic cytidine derivative developed in the 1970s and is used for the treatment myelodysplastic syndrome (in the following sometimes also referred to as MDS) and leukemia. It acts as a mimic of cytidine, a building block of RNA and is classified as a pyrimidine antimetabolite.

Azacitidine is commonly administered by intravenous infusion, intravenous injection or subcutaneous injection. After being absorbed, azacitidine is subsequently converted into the respective mono-, di- and triphosphate and incorporated into RNA strands, which disrupts proper RNA functioning and impairs tRNA cytosine-5-methyltransferase activity.

In addition, azacitidine's primary mode of action is the inhibition of the enzyme DNA methyltransferase (DNMT-1)—the enzyme responsible for methylation of DNA—by azacitidine's metabolite 5-aza-2′-deoxycytidine-triphosphate. 5-Aza-2′-deoxy-cytidine-triphosphate inhibits the enzyme DNA methyltransferase by forming an irreversible, covalent bond with the enzyme DNA methyltransferase. Consequently, DNA methylation can no longer be reproduced in DNA daughter strands and previously silenced cell cycle-regulating and tumor-suppressing genes that were initially silenced due to hypermethylation are reactivated, resulting in an antitumour effect.

Decitabine (IUPAC name 5-aza-2′-deoxycytidine) is a close derivative of azacitidine and functions in a similar mode of action by inhibiting the enzyme DNMT-1. The chemical formulae of azacitidine (1) and decitabine (2) are depicted below.

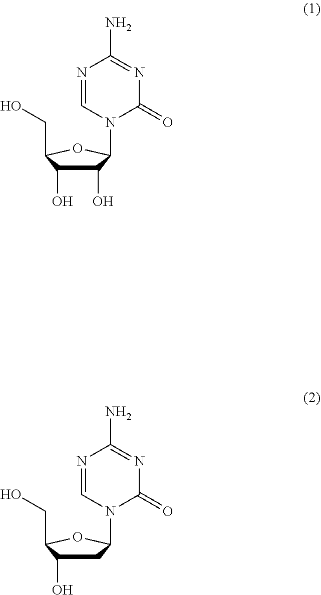

Azacitidine quickly degrades in most solvents at room temperature by several different degradation mechanisms. Among others, the nucleophilic attack on the N5-nitrogen and ring-opening to form N-(formylamidino)-N′-β-d-ribofuranosylurea, which can further degrade to 1-β-d-ribofuranosyl-3-guanylurea, is one degradation pathway, as summarized in Argami et al., Talanta, volume 74, 2007, pp. 176-182. Due to its instability in solution, azacitidine is provided as a dry 100 mg lyophilizate that is suspended in water as a 25 mg/mL suspension prior to subcutaneous (s.c.) use or is solubilized in water as a 10 mg/mL solution (the solubility of azacitidine being 13.4 mg/mL at 25° C. in water) prior to subcutaneous use. In view of the recommended dosage of azacitidine, most doses require more than one 100 mg lyophilizate vial of azacitidine so that the suspension or dissolution step needs to be performed several times for one treatment dose.

Thus, one drawback of the azacitidine formulations (or decitabine formulations) of the prior art is the need to prepare the formulation in situ before treatment. This requires vigorous shaking to prepare a solution for intravenous (i.v.) administration or a uniform suspension for s.c. administration and visual inspection of the formulation prior to administration. If the suspension or solution is not uniform it must be discarded, and the process must be repeated. Accordingly, the reconstitution procedure is labor and time intensive and can further result in unnecessary dosing errors as well as the risk of potential contamination. Specifically for s.c. suspensions refrigerated storage after reconstitution often leads to formation of API agglomerates which are hard to re-suspend. In addition, it is only possible to administer the azacitidine formulation at low concentrations of 10 mg/mL or 25 mg/mL, thus a large injection volume is required of often more than 4 mL, thus requiring more than one reconstitution step and more than one injection. Such large injection volumes can result in a high incidence of injection site reaction in patients receiving azacitidine by s.c. administration, such as painful lesions, rash, inflammation, pruritus, erythema and subsequent soft tissue infections. In rare cases, the resulting infections have been reported to even lead to death (see the European public assessment report of Vidaza®). These risks are further increased for administration volumes above 4 mL, which require more than one injection. Thus, another drawback of the azacitidine formulations (or decitabine formulations) of the prior art is the high incidence of injection site reactions, in particular for s.c. administration.

Additionally, the reconstitution step for forming a suspension can lead to agglomeration of particles resulting in a dosing that is difficult and unpredictable as the particles are distributed unevenly in the suspension. Due to re-agglomeration of the particles after the reconstitution step, the suspension should not be stored for more than 1 hour at 25° C. after reconstitution (see the Summary of Product Characteristics (SmPC) of Vidaza®). This results in a logistically cumbersome administration process, as the reconstituted formulation must be brought and administered to the patient within 1 hour after reconstitution at a compounding station.

Therefore, it was an object of the present invention to provide a pharmaceutical azacitidine or decitabine formulation that is a ready-to-use formulation while maintaining high stability at room temperature. In addition, it was another object of the present invention to provide a pharmaceutical azacitidine or decitabine formulation that is highly concentrated, therefore minimizing the chances of adverse reactions at the injection site.

### SUMMARY OF THE INVENTION

These objects have surprisingly been solved by the present parenteral pharmaceutical composition comprising azacitidine and dimethyl sulfoxide, wherein the pharmaceutical composition comprises azacitidine at a concentration of from 40 mg/mL to 340 mg/mL.

It has surprisingly been found that a stable, highly concentrated and ready-to-use formulation can be provided by dissolving azacitidine in dimethyl sulfoxide (DMSO). This finding was in particular unexpected as DMSO is rarely used in pharmaceutical compositions aside from transdermal therapeutic systems where the concentration of DMSO does not exceed 10 wt.-%, and further the nucleophilicity of nucleophiles is increased in polar aprotic solvents, such as DMSO. Therefore, it was believed that degradation due to nucleophilic attacks is increased in DMSO and it was unexpected that the stability of azacitidine instead increases in DMSO.

The solubility of bulk (i.e. non-pharmaceutical grade) azacitidine or decitabine with a purity above 95% in bulk (i.e. non-pharmaceutical grade) DMSO was expected to be approximately 30 mg/mL (see e.g. product information leaflet from e.g. the Cayman Chemical Company). Thus, it was unexpected that the pharmaceutical composition of the present invention comprising azacitidine and DMSO or decitabine and DMSO can be prepared in a concentration of above 30 mg/mL.

In addition, pharmaceutical compositions are often stored and transported in refrigerated conditions or simply kept at ambient temperatures below 18.5° C. and it is important that a ready-to-use formulation does not solidify. Solidification can occur due to freezing of the formulation or due to precipitation of the active ingredient due to a decreased solubility at lower temperatures. Solidification can be detrimental to stability, uniformity as well as the purpose of ready-to-use formulations, i.e. being ready-to-use. However, pure DMSO solidifies at 18.5° C. Therefore, it was unexpected that the pharmaceutical composition of the present invention comprising azacitidine and DMSO (or decitabine and DMSO) does not solidify at temperatures below 18.5° C., such as 13.5° C. or 16° C.

Furthermore, it has surprisingly been found that a stable infusion composition comprising the pharmaceutical composition and an infusion solution can be prepared, wherein no solidification or decomposition occurs, in particular at a temperature of 0° C. to 15° C.

In another aspect, the invention relates to an injection syringe, a container or a vial containing the pharmaceutical composition for single or multiple dosing.

In another aspect, the present invention relates to the pharmaceutical composition of the present invention for use in the treatment of myelodysplastic syndrome, acute myelomonocytic leukemia or chronic myelomonocytic leukemia.

In another embodiment, the present invention relates to a kit of parts comprising the pharmaceutical composition of the present invention in a vial and an injection device.

In yet another embodiment, the present invention relates to a method for providing the pharmaceutical composition of the present invention, comprising the steps of

(a) providing azacitidine; and

(b) mixing the azacitidine with dimethyl sulfoxide to provide the pharmaceutical composition.

In yet another embodiment, the present invention relates to an infusion composition comprising the pharmaceutical composition of the present invention and an infusion solution.

### BRIEF DESCRIPTION OF THE DRAWINGS

FIG. 1 shows the turbidity measurement of a 200 mg/mL azacitidine solution in DMSO as described in Example 2.

### DETAILED DESCRIPTION

The following definitions are relevant in connection with the embodiments of the present invention.

The term “parenteral” preferably refers to a composition that is not administered orally. In particular, the parenteral composition is suitable for injection or infusion, especially for administration by intravenous injection, intravenous infusion, intramuscular injection and/or subcutaneous injection.

The term “pharmaceutical” preferably refers to compositions wherein the active ingredient and/or the dimethyl sulfoxide fulfils all necessary pharmaceutical standards/monographs of the USP-NF (United States Pharmacopeia National Formulary) and/or Ph. Eur. (European Pharmacopoeia) with regard to the purity of the active ingredient and/or amount of pathogens and/or bacterial contamination.

Accordingly, the dimethyl sulfoxide used in the present invention preferably fulfills the requirements of Ph. Eur. monograph 9.0, 0763. Further, the term “pharmaceutical composition” encompasses a composition fulfilling Ph. Eur. standards/monographs 9.0/2.06.08.00 and 9.3/2.06.14.00, which relate to the measurement and amounts of pyrogens and bacterial endotoxins, respectively.

The term “azacitidine” preferably refers to the azacitidine free base. However, azacitidine may also refer to pharmaceutically acceptable salts, polymorphs, solvates and mixtures thereof. The term “pharmaceutically acceptable salts” refers to the addition salt of the free base with hydrochloric acid, hydrobromic acid, boric acid, phosphoric acid, acetic acid, citric acid, fumaric acid, maleic acid, malic acid, malonic acid, oxalic acid, succinic acid, tartaric acid, p-toluenesulfonic acid, trifluoromethane-sulfonic acid or trifluoroacetic acid. The term “solvate” refers to azacitidine complexed with a solvent molecule other than water that is bound by intermolecular forces in the crystal lattice of the azacitidine (before dissolution in DMSO). Likewise, the term “decitabine” preferably refers to the decitabine free base. However, decitabine may also refer to pharmaceutically acceptable salts, polymorphs, solvates and mixtures thereof.

The meaning of the term “comprising” is to be interpreted as encompassing all the specifically mentioned features as well as optional, additional and unspecified ones, whereas the term “consisting of” only includes the features as specified. Therefore, “comprising” includes as a limiting case the composition specified by “consisting of”.

The term “wt.-%” refers to the amount of the respective ingredient by weight based on the total amount of the composition unless noted otherwise.

Preferred embodiments according to the invention are defined hereinafter. The preferred embodiments are preferred alone or in combination. Further, it is to be understood that the following preferred embodiments refer to all aspects of the present invention, i.e. the pharmaceutical composition, the compound for use, the kit of parts and the method for providing the pharmaceutical composition.

In an embodiment, the pharmaceutical composition of the present invention comprises azacitidine and dimethyl sulfoxide or decitabine and dimethyl sulfoxide In an embodiment, the pharmaceutical composition of the present invention consists of azacitidine and dimethyl sulfoxide or decitabine and dimethyl sulfoxide. In an embodiment, the pharmaceutical composition of the present invention consists of azacitidine and dimethyl sulfoxide. Thus, it is preferred that the pharmaceutical composition does not comprise any excipients, buffers, additional antimicrobial agents, surfactants or solubility enhancers.

In another embodiment, the pharmaceutical composition comprises one or more surfactant(s). Surfactants can prevent aggregation of molecules in the formulation. Non-limiting examples of surfactants are polysorbates (Tween®), such as polysorbate 20, polysorbate 40, polysorbate 60, polysorbate 80 and polysorbate 120, polyoxyl castor oils, poloxamers and lecithins. In a preferred embodiment, the pharmaceutical composition does not comprise one or more surfactants. It has surprisingly been found that the stability of the pharmaceutical composition can be maintained, and aggregation of particles can be prevented even in the absence of surfactants.

In an embodiment, the pharmaceutical composition is essentially free of water. The term essentially free of water refers to a pharmaceutical composition that contains less than 0.8 wt.-% water, preferably less than 0.5 wt.-% water or more preferably less than 0.12 wt.-% water, or particularly preferred less than 0.10 wt.-% water. As the water content may potentially vary during storage, it is preferred that the pharmaceutical composition contains less than the afore-mentioned water content at any point during the storage period. The water content can be measured by Karl Fischer titration with e.g. a Mettler DL 18 titrator.

It is surprising that a ready-to-use formulation can be provided containing DMSO, in particular in the absence of water, that does not solidify when refrigerated or cooled below 18.5° C. or when the ambient temperature drops below 18.5° C., such as to 16° C. or 13.5° C. The freezing point of DMSO can be substantially reduced by the presence of co-solvents, such as water. Therefore, it was surprising that a substantial freezing point reduction is achieved even in the absence of water, as binary mixtures of water and DMSO have substantially reduced freezing points. It has surprisingly been found that the freezing point of the pharmaceutical composition essentially free of water comprising an approximately 0.6 molar concentration of azacitidine (150 mg/mL) in DMSO is reduced to below 8° C. This freezing point reduction is significantly larger than expected from Bladgen's law of freezing point depression. The cryoscopic constant of DMSO is approximately 4.0 K kg/mol (see George et al., Anal. Chem., 1966, 38, 9, 1285-1286: “Cryoscopic Molecular Weight Determinations Using Dimethyl Sulfoxide as the Solvent”). Therefore, for a 150 mg/mL solution of azacitidine in DMSO, a freezing point reduction of approximately 2.5° C. is to be expected, which would make this solution unsuitable to be stored or transported at temperatures of 16° C. or lower. In view of the above, it has surprisingly been found that the freezing point reduction of a solution of azacitidine in DMSO that is essentially free of water is indeed larger than expected, making this pharmaceutical composition suitable to be stored and transported at reduced temperatures.

In another embodiment, the pharmaceutical composition is a solution. This allows for the administration by intravenous infusion or injection as well as subcutaneous injection. However, it is also possible to provide the pharmaceutical composition as a suspension. Said suspension preferably might be used for subcutaneous injection.

In an embodiment, the composition is sterile in accordance with Ph. Eur. 9.0/2.06.01.00 and/or 9.00/5.01.01.00.

In an embodiment, the composition is sterile, i.e. it contains less than 10⁻⁴ wt.-% of nonsterile material, such as microbial contamination. Sterility of the composition can be ensured by a number of processes, such as thermal, chemical, radiation and filtration processes. In the present invention, it is preferred that a nonthermal process is employed. Thus, it is preferred that sterilization is ensured by either gamma ray or UV light irradiation or by filtration through a suitable membrane filter. Suitable filters are known to the skilled person and are described in “The Theory and Practice of Industrial Pharmacy”, Lachman et al., 3^{rd} edition, Varghese Publishing house. Examples include cellulose filters, Nylon 66, polytetrafluoroethylene with polyethylene or polypropylene substrate. The membranes of the filters usually have a nominal pore size of 0.22 μm or less, preferably 0.20 μm or less, more preferably 0.10 μm or less.

In an embodiment, the pharmaceutical composition has a storage stability of 1 to 5 years, 1.5 to 4 years or 2 to 3 years at 25° C., preferably of 1.5 to 3 years at 25° C. The term “storage stability” refers to a composition, wherein azacitidine or decitabine does not change, deteriorate, react or decompose over the prescribed time period by more than 5 wt.-%, more than 4 wt.-%, more than 3 wt.-%, more than 2 wt.-%, more than 1.5 wt.-%, more than 1 wt.-%, more than 0.5 wt.-% or more than 0.1 wt.-% based on the total amount of the respective component. It is preferred that the term storage stability refers to a composition wherein the azacitidine or decitabine does not change, deteriorate, react or decompose over the prescribed time period by more than 5 wt.-%.

In a preferred embodiment, the pharmaceutical composition has a storage stability at 25° C. of up to 1 year, up to 1.5 years or up to 2 years. In an embodiment, the pharmaceutical composition has a storage stability of up to 10 months, up to 12 months or up to 14 months at 40° C.

In an embodiment, the pharmaceutical composition further comprises an additional solvent, preferably the additional solvent is selected from dimethylacetamide (DMA), dimethyl isosorbide (DMI) and propylene carbonate. Such a solvent mixture can be advantageous for further increasing the stability of the pharmaceutical composition while maintaining a high solubility of azacitidine. In an embodiment, the pharmaceutical composition comprises azacitidine or decitabine, dimethyl sulfoxide and an additional solvent selected from the group consisting of dimethylacetamide, dimethyl isosorbide, propylene carbonate and mixtures thereof.

In an embodiment, the pharmaceutical composition consists of azacitidine or decitabine, dimethyl sulfoxide and an additional solvent selected from the group consisting of dimethylacetamide, dimethyl isosorbide, propylene carbonate and mixtures thereof.

In an embodiment, the pharmaceutical composition comprises azacitidine at a concentration of from 40 mg/mL to 340 mg/mL, of from 50 mg/mL to 250 mg/mL, of from 60 mg/mL to 240 mg/mL, of from 70 mg/mL to 230 mg/mL, of from 80 mg/mL to 220 mg/mL, of from 90 mg/mL to 210 mg/mL, of from 100 mg/mL to 200 mg/mL, of from 110 mg/mL to 190 mg/mL, of from 120 mg/mL to 180 mg/mL, of from 130 mg/mL to 170 mg/mL, of from 140 mg/mL to 160 mg/mL, or of from 150 mg/mL to 160 mg/mL, preferably at a concentration of from 100 mg/mL to 300 mg/mL. In a preferred embodiment, the pharmaceutical composition comprises azacitidine at a concentration of from 50 mg/mL to 250 mg/mL, from 100 mg/mL to 220 mg/mL or from 110 mg/mL to 190 mg/mL. In an even more preferred embodiment, the pharmaceutical composition comprises azacitidine at a concentration of from 150 mg/mL to 200 mg/mL.

In an even more preferred embodiment, the pharmaceutical composition comprises azacitidine at a concentration of 150 mg/mL, of 155 mg/mL, of 160 mg/mL, of 165 mg/mL, of 170 mg/mL, of 175 mg/mL, of 180 mg/mL, of 185 mg/mL, of 190 mg/mL, of 195 mg/mL or of 200 mg/mL.

In an embodiment, the pharmaceutical composition comprises decitabine at a concentration of from 40 mg/mL to 90 mg/mL.

In a particularly preferred embodiment, the present invention relates to a parenteral pharmaceutical composition comprising azacitidine and dimethyl sulfoxide, wherein the pharmaceutical composition is essentially free of water, and wherein the pharmaceutical composition comprises azacitidine at a concentration of 150 mg/mL to 200 mg/mL.

In a particularly preferred embodiment, the present invention relates to a parenteral pharmaceutical composition comprising azacitidine and dimethyl sulfoxide, wherein the pharmaceutical composition contains less than 0.5 wt.-% water, and wherein the pharmaceutical composition comprises azacitidine at a concentration of 150 mg/mL to 200 mg/mL.

In a particularly preferred embodiment, the present invention relates to a parenteral pharmaceutical composition consisting of azacitidine and dimethyl sulfoxide, wherein the pharmaceutical composition comprises azacitidine at a concentration of 150 mg/mL to 200 mg/mL.

In an embodiment, the pharmaceutical composition is a ready-to-use composition for injection or infusion or a concentrate. The concentrate may be useful for administration by infusion. Thus, the pharmaceutical composition of the present invention does not need to be manually prepared in situ and can be used as is without an additional reconstitution step.

In an embodiment, the invention relates to an injection syringe, a container or a vial containing the pharmaceutical composition of the present invention. The container or vial can be made of glass or plastic. The composition may be removed from the container or vial by either breaking the vial or container at a predetermined breaking point or by providing the vial or container with a closure, i.e. a rubber septum/stopper, which can be punctured by a needle. Examples for suitable plastic materials comprise polyethylene, polypropylene, polyvinyl chloride, polycarbonate, polyamide polystyrene and polytetrafluoroethylene. These plastic materials may contain further additives, such as colorants, stabilizers and antioxidants. Examples for glass are soda-lime glass and boiosilicate. Examples of suitable rubber septa/stoppers are butyl rubbers, neoprene rubber, silicone rubbers. In order to prevent moisture absorption, the rubber septum/stopper may be coated by a hydrophobic coating agent, such as silicone polymers or may contain a desiccant. It is particularly preferred that the container or vial is filled using a blow-fill-seal (BFS) technology wherein the container or vial is molded, aseptically filled and sealed in a continuous process.

The use of pre-filled syringes or cartridges for syringe systems reduces the degree of manipulation required and therefore further facilitates easy administration.

In a particularly preferred embodiment, each injection syringe, cartridge, container or vial has a uniform content of azacitidine in accordance with Ph. Eur. 9.00/2.09.40.00.

In a preferred embodiment, the injection syringe, cartridge, container or vial contains a nominal volume of 0.2 mL, 0.3 mL, 0.4 mL, 0.5 mL, 1.0 mL, 1.5 mL, 2.0 mL, 5.0 mL, 7.5 mL, 10 mL, 12.5 mL, 15 mL, 15 mL, 17.5 mL or 20 mL of the pharmaceutical composition. It is even more preferred that the injection syringe, cartridge, container or vial is a single-dose injection syringe, container or vial. The term “nominal” volume refers to the volume as intended but the actual volume may deviate by an amount of ±5 volume % from the nominal volume. In a preferred embodiment, the term “nominal” volume in each vial complies with Ph. Eur. 9.00/2.09.17.00 regarding the amount of extractable volume.

In a preferred embodiment, the pharmaceutical composition is contained in a vial. It is even more preferred that the pharmaceutical composition is contained in a multi-dose vial. A multi-dose vial can contain sufficient composition for administrations over several days. It is preferred that the multi-dose vial contains the pharmaceutical composition in an amount sufficient for at least seven days. The multi-dose vial may contain the pharmaceutical composition in an amount of from 1.0 mL to 50 mL, preferably in an amount of from 5.0 to 10 mL.

In a preferred embodiment, the invention relates to a pharmaceutical composition comprising azacitidine and DMSO in a multi-dose vial, wherein the pharmaceutical composition is essentially free of water, wherein the pharmaceutical composition comprises azacitidine at a concentration of 150 mg/mL to 200 mg/mL, and wherein the multi-dose vial contains 1.0 to 50 mL of the pharmaceutical composition.

In a preferred embodiment, the invention relates to a pharmaceutical composition comprising azacitidine and DMSO in a multi-dose vial, wherein the pharmaceutical composition contains less than 0.5 wt.-% water, wherein the pharmaceutical composition comprises azacitidine at a concentration of 150 mg/mL to 200 mg/mL, and wherein the multi-dose vial contains 1.0 to 50 mL of the pharmaceutical composition.

In a preferred embodiment, the invention relates to a pharmaceutical composition consisting of azacitidine and dimethyl sulfoxide in a multi-dose vial, wherein the pharmaceutical composition comprises azacitidine at a concentration of 150 mg/mL to 200 mg/mL, and wherein the multi-dose vial contains 1.0 to 50 mL of the pharmaceutical composition.

In an embodiment, the invention relates to the pharmaceutical composition for use in the treatment of myelodysplastic syndrome, acute myelomonocytic leukemia or chronic myelomonocytic leukemia, preferably for use in the treatment of myelodysplastic syndrome.

In particular, the myelodysplastic syndrome may be any of the following types according to the French-American-British classification: refractory anemia (RA), refractory anemia with ringed sideroblasts (RARS), refractory anemia with excess blasts (RAEB I), refractory anemia with excess blasts in transformation (RAEB II or RAEB-T) and chronic myelomonocytic leukemia (CMML).

In an embodiment, the pharmaceutical composition is administered systemically. The term systemically refers to the delivery in the circulatory system of the subject and thus inherently affects its whole body. Examples of systemic administration include intramuscular (i.m.), intravenous (i.v.), intradermal (i.d.), transdermal (t.d.) and subcutaneous (s.c.). In an embodiment, the pharmaceutical composition is administered by subcutaneous (s.c.) or by intravenous (i.v.) injection or infusion.

In a particularly preferred embodiment, the present invention relates to a parenteral pharmaceutical composition comprising azacitidine and dimethyl sulfoxide for use in the treatment of myelodysplastic syndrome, acute myelomonocytic leukemia or chronic myelomonocytic leukemia, preferably for use in the treatment of myelodysplastic syndrome, wherein the pharmaceutical composition is essentially free of water, wherein the pharmaceutical composition comprises azacitidine at a concentration of 150 mg/mL to 200 mg/mL, and wherein the pharmaceutical composition is administered by intravenous (i.v.) injection or infusion.

In a particularly preferred embodiment, the present invention relates to a parenteral pharmaceutical composition comprising azacitidine and dimethyl sulfoxide for use in the treatment of myelodysplastic syndrome, acute myelomonocytic leukemia or chronic myelomonocytic leukemia, preferably for use in the treatment of myelodysplastic syndrome, wherein the pharmaceutical composition contains less than 0.5 wt.-% water, wherein the pharmaceutical composition comprises azacitidine at a concentration of 150 mg/mL to 200 mg/mL, and wherein the pharmaceutical composition is administered by intravenous (i.v.) injection or infusion.

In a particularly preferred embodiment, the present invention relates to a parenteral pharmaceutical composition consisting of azacitidine and dimethyl sulfoxide for use in the treatment of myelodysplastic syndrome, acute myelomonocytic leukemia or chronic myelomonocytic leukemia, preferably for use in the treatment of myelodysplastic syndrome, wherein the pharmaceutical composition comprises azacitidine at a concentration of 150 mg/mL to 200 mg/mL, and wherein the pharmaceutical composition is administered by intravenous (i.v.) injection or infusion.

In a particularly preferred embodiment, the present invention relates to a parenteral pharmaceutical composition consisting of azacitidine and dimethyl sulfoxide for use in the treatment of myelodysplastic syndrome, wherein the pharmaceutical composition comprises azacitidine at a concentration of 150 mg/mL to 200 mg/mL, and wherein the pharmaceutical composition is administered by intravenous (i.v.) infusion.

In case the pharmaceutical composition is administered by intravenous (i.v.) infusion, the pharmaceutical composition is mixed with an infusion solution. Suitable infusion solutions are for example electrolyte solutions selected from the group consisting of 0.9 wt.-% aqueous NaCl solution, Ringer's solution, Ringer's lactate solution and Ringer's acetate solution. Thus, it is preferred that the pharmaceutical composition is injected into an infusion solution to form a mixture of infusion solution and pharmaceutical composition which is administered to a patient. Administration of this mixture can be performed through a peripheral venous catheter. Preferably, the infusion solution may have a volume of 25 to 500 mL, preferably of 50 to 100 mL. It is preferred that the infusion solution is 0.9 wt.-% aqueous NaCl solution.

The pharmaceutical composition for use in the treatment of myelodysplastic syndrome, acute myelomonocytic leukemia or chronic myelomonocytic leukemia can be administered by i.v. infusion by preparing an infusion composition comprising the pharmaceutical composition of the present invention and an infusion solution that is administered.

In a particularly preferred embodiment, the pharmaceutical composition for use in the treatment of myelodysplastic syndrome comprises azacitidine and DMSO, wherein the pharmaceutical composition is administered as an infusion composition.

It is preferred that the infusion composition is prepared with an infusion solution having a temperature of 0° C. to 15° C. so that the infusion composition has a temperature of 0° C. to 15° C. This infusion composition may be administered directly or may be administered once the infusion composition has a temperature of above 15° C., preferably a temperature around room temperature, i.e. about 25° C.

In an embodiment, the pharmaceutical composition is administered at an amount of azacitidine of 60 to 100 mg/m²/day, preferably at an amount of 75 mg/m²/day, for seven days every 28 days. It is recommended that the patient is treated for a minimum of 6 cycles and treatment should be continued for as long as the patient benefits from the treatment. When the dosage is based on m², i.e. the body surface area (BSA) of the patient, the body surface area can be estimated based on the height and weight of the patient according to the Du Bois or according to the Mosteller formula.

In an embodiment, the pharmaceutical composition is administered at an amount of decitabine of 10 to 30 mg/m²/day, preferably at an amount of 20 mg/m²/day, for five consecutive days every 28 days. It is recommended that the patient is treated for a minimum of 4 cycles and treatment should be continued for as long as the patient benefits from the treatment.

In an embodiment, the invention relates to a kit of parts comprising the pharmaceutical composition in a vial, cartridge or container and an injection device.

In an embodiment, the invention relates to a method for preparing the pharmaceutical composition, comprising the steps of
(a) providing azacitidine or decitabine; and
(b) mixing the azacitidine or the decitabine with dimethyl sulfoxide to provide the pharmaceutical composition.

In an embodiment, the method further comprises the step of
(c) sterilization of the pharmaceutical composition by gamma ray irradiation and/or by filtration through a suitable membrane filter. It is preferred that sterilization takes place by filtration.

It is preferred that the method further comprises the step of
(d) filling the pharmaceutical composition into a syringe, cartridge, vial or container and sealing the syringe, cartridge, vial or container. It is preferred that a rubber stopper and a crimp cap is used for sealing the vial or container.

It is preferred that all steps are performed under aseptic conditions. It is further preferred that the method is a continuous method.

In a preferred embodiment, the method for preparing the pharmaceutical composition comprises the steps of
(a) providing azacitidine or decitabine;
(b) mixing the azacitidine or the decitabine with dimethyl sulfoxide; and
(c) sterilization of the pharmaceutical composition by filtration through a suitable membrane filter having a nominal pore size of 0.22 μm or less,

wherein all steps are performed under aseptic conditions to provide a sterile pharmaceutical composition in accordance with Ph. Eur. 9.0/2.06.01.00 and/or 9.00/5.01.01.00.

In a preferred embodiment, the method for preparing the pharmaceutical composition comprises the steps of
(a) providing azacitidine or decitabine;
(b) mixing the azacitidine or the decitabine with dimethyl sulfoxide; and
(c) sterilization of the pharmaceutical composition by filtration through a suitable membrane filter having a nominal pore size of 0.22 μm or less; and
(d) filling the pharmaceutical composition into a syringe, cartridge, vial or container and sealing the syringe, cartridge, vial or container,

wherein all steps are performed under aseptic conditions to provide a sterile pharmaceutical composition in accordance with Ph. Eur. 9.0/2.06.01.00 and/or 9.00/5.01.01.00.

It is preferred that one or more steps of preparing the pharmaceutical composition of the invention, preferably all steps, are performed under an inert atmosphere. The term “inert atmosphere” refers to an inert gas, such as nitrogen or argon.

The manufacture of sterile pharmaceutical compositions is i.a. described in “Rule and Guidance for Pharmaceutical Manufacturers and Distributors” (The Orange Guide) by the Medicines and Healthcare products Regulatory Agency, published by Pharmaceutical Press, 2017.

In an embodiment, the present invention relates to an infusion composition comprising the pharmaceutical composition of the present invention and an infusion solution.

The infusion composition is obtainable by mixing the pharmaceutical composition of the present invention and an infusion solution. Preferably, the infusion solution has a temperature of 0° C. to 15° C., 1° C. to 12° C., 2° C. to 10° C. or 3° C. to 5° C. It is preferred that the infusion solution has a temperature below 23° C., 22° C., 21° C., 20° C., 19° C., 18° C., 17° C., 16° C., 15° C., 14° C., 13° C., 12° C., 11° C. or 10° C.

Consequently, it is preferred that the infusion composition has a temperature of 0° C. to 15° C., 1° C. to 12° C., 2° C. to 10° C. or 3° C. to 5° C. It is preferred that the infusion composition has a temperature below 23° C., 22° C., 21° C., 20° C., 19° C., 18° C., 17° C., 16° C., 15° C., 14° C., 13° C., 12° C., 11° C. or 10° C. This temperature relates to the temperature before and during administration of the infusion composition. However, the infusion composition may also be prepared at this temperature and then be allowed to reach a higher temperature before administration to a patient in need thereof.

It is generally preferred that the infusion composition is kept at a low temperature, as this increases the stability of the azacitidine or decitabine. However, administration at a higher temperature is possible to make the administration more comfortable for the patient.

It is preferred that the concentration of the azacitidine or the decitabine is 0.5 to 50 mg/mL, 1 to 10 mg/mL, 1.5 to 4 mg/mL or 2 to 3 mg/mL.

The infusion composition and/or the pharmaceutical composition of the present invention is essentially free of impurities. The term essentially free of impurities refers to a composition that contains less than 0.8 wt.-% impurities, preferably less than 0.5 wt.-% impurities or more preferably less than 0.12 wt.-% impurities or particularly preferred less than 0.10 wt.-% impurities. The term “impurities” refers to any degradation product of the azacitidine or the decitabine. The purity of the active ingredient may be measured according to pharmaceutical standards/monographs of the USP-NF (United States Pharmacopeia National Formulary) and/or Ph. Eur. (European Pharmacopoeia) and the impurities can be identified by HPLC (high performance liquid chromatography).

### EXAMPLES

### Example 1: Stability Testing

A 250 mg/mL solution of azacitidine (TEVA Sicor) in DMSO (Merck; Ph. Eur. grade quality) with a water content of less than 0.1 wt.-% was prepared and filled into (moisture proof) vials, which were subsequently sealed using rubber stoppers. The stability of the azacitidine was measured at storage temperatures of 25° C. and 40° C. after 0 weeks, 1 week, 2 weeks, 4 weeks, 2 months, 3 months and 6 months after sealing the solution in the vial. The amount of azacitidine was analyzed using high performance liquid chromatography (HPLC) and comparing the integral of the azacitidine peak with the integral of a freshly prepared standard solution containing a predetermined amount of azacitidine.

HPLC measurements were carried out using an Agilent 1200 system (or equivalent). For detection, a UV detector (235 nm) was used. A YMC Hydrosphere C18 (250 mm*4.6 mm*5000 mm) column and a flow rate of 1.0 mL/min and a gradient of aq. pH 7 buffer solution (containing disodium hydrogen phosphate, orthophosphoric acid and ammonium formate)/acetonitrile from 100:0 to 40:60 were employed. The results of the stability test are shown in Table 1.

TABLE 1
Amount of Azacitidine during storage at 25° C. and 40° C.:

| Day(s) after beginning | %* of azacitidine | %* of azacitidine |
|---|---|---|
| of storage | at 25° C. | at 40° C. |
| 0 | 100 | 100 |
| 7 | 100 | 100 |
| 14 | 100 | 100 |
| 21 | 100 | 100 |
| 28 | 100 | 100 |
| 56 | 100 | 99 |
| 98 | 100 | 96 |
| 182 | 100 | 94 |

*value based on the amount of azacitidine/amount of azacitidine at week 0.

It was confirmed that the composition containing azacitidine in dimethyl sulfoxide is stable for at least 182 days (6 months) at 25° C. Predicted stability is at least 18 months at 25° C. The water content was measured throughout the 6 months by Karl-Fischer titration and was below 0.1 wt.-% at all times.

### Example 2: Solidification-Stability

Crystallization/freezing of the solvent was determined by turbidity measurements using a Crystal 16 crystallizer by Technobis Crystallization Systems. 0.5 mL of an azacitidine solution in DMSO (200 mg/mL) was magnetically stirred with 500 rpm.

The following temperature program was applied:

room temperature (around 20° C.) to 13° C. with −0.1 K/min

hold at 13° C. for 96 h

13° C. to 12° C. with −0.1 K/min

hold at 12° C. for 96 h

12° C. to 11° C. with −0.1 K/min

hold at 11° C. for 96 h

11° C. to 23° C. with 0.1 K/min

hold at 23° C. for 24 h.

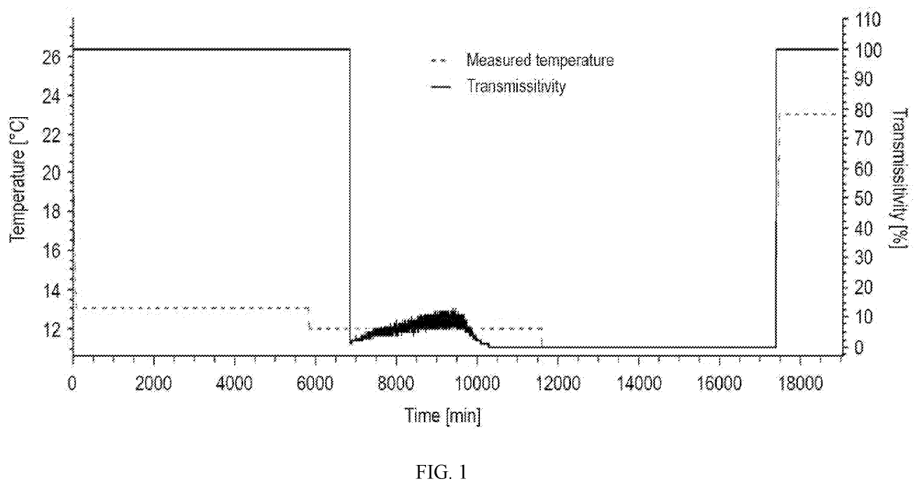

It was found that solidification of the 200 mg/mL azacitidine formulation in DMSO took place at around 13° C., as can inferred from FIG. 1.

In a similar way, crystallization/freezing of solvent was determined for a 150 mg/mL azacitidine solution in DMSO. It was found that solidification of the 150 mg/mL azacitidine formulation in DMSO took place at around 8° C.

### Example 3: Dilution Stability for i.v. Infusion

A 250 mg/mL solution of azacitidine (TEVA Sicor) in DMSO (Merck; Ph. Eur. grade quality) with a water content of less than 0.1 wt.-% was prepared. Subsequently 0.40 mL of the 250 mg/mL azacitidine in DMSO solution was diluted with 40 mL of a 0.9 wt.-% aqueous NaCl solution at 25° C.

No precipitation could be observed during the dilution. This lack of precipitation shows that the azacitidine solution in DMSO is suitable as a concentrate for administration by i.v. infusion.

### Example 4: Temperature-Dependent Dilution Stability

A 250 mg/mL solution of azacitidine (TEVA Sicor) in DMSO (Merck; Ph. Eur. grade quality) with a water content of less than 0.1 wt.-% was prepared. In addition, a 0.9 wt.-% aqueous NaCl (Merck, Ph. Eur. grade quality) solution was prepared. Subsequently 0.40 mL of the 250 mg/mL azacitidine in DMSO solution was diluted with 40 mL of the 0.9 wt.-% aqueous NaCl solution, the 0.9 wt.-% aqueous NaCl solution having a temperature of 23-24° C. (Example 4A) or a temperature of 3-4° C. (Example 4B).

Similar to Example 3, no precipitation could be observed during the dilution of Example 4A. During the dilution of Example 4B, minor precipitation could be observed during the dilution process. The precipitation, however, redissolved within a short period of time. This shows that at both temperatures, a suitable infusion composition for administration by i.v. infusion can be prepared.

Subsequently, the chemical stability of the azacitidine was measured for Example 4A (kept at 23-24° C.) and Example 4B (kept at 3-4° C.). The amount of azacitidine was measured by HPLC.

The results of the stability test are shown in Table 2.

TABLE 2
Amount of azacitidine in an infusion solution at different temperatures:

| Hour(s) after | %* of azacitidine in | %* of azacitidine |
|---|---|---|
| preparation | Example 4A (23-24° C.) | Example 4B (3-4° C.) |
| 0 | 100 | 100 |
| 0.5 | 89 | 99 |
| 1 | 81 | 97 |
| 3 | 66 | 92 |
| 20 | 49 | 68 |
| 96 | 41 | 53 |

*value based on the amount of azacitidine/amount of azacitidine at 0 h.

1. A parenteral pharmaceutical composition comprising azacitidine and dimethyl sulfoxide, wherein the pharmaceutical composition comprises azacitidine at a concentration of from 40 mg/mL to 340 mg/mL.

2. The pharmaceutical composition according to claim 1, wherein the pharmaceutical composition is essentially free of water.

3. The pharmaceutical composition according to claim 1, wherein the pharmaceutical composition is a solution.

4. The pharmaceutical composition according to claim 1, wherein the composition is sterile in accordance with European Pharmacopoeia 9.0/2.06.01.00 and/or 9.00/5.01.01.00.

5. The pharmaceutical composition according to claim 1 further comprising a solvent selected from dimethylacetamide, dimethylisosorbid and propylene carbonate.

6. The pharmaceutical composition according to claim 1, wherein the pharmaceutical composition comprises azacitidine at a concentration of from 150 to 200 mg/mL.

7. The pharmaceutical composition according to claim 1, wherein the pharmaceutical composition is a ready-to-use composition or a concentrate.

8. An injection syringe, a cartridge, a container or a vial containing the pharmaceutical composition according to claim 1 for single or multiple dosing.

9. The pharmaceutical composition according to claim 1 for use in the treatment of myelodysplastic syndrome, acute myelomonocytic leukemia or chronic myelomonocytic leukemia.

10. The pharmaceutical composition for use according to claim 9, wherein the pharmaceutical composition is administered by subcutaneous or intravenous injection.

11. The pharmaceutical composition for use according to claim 9, wherein the pharmaceutical composition is administered at an amount of azacitidine of 60 to 80 mg/m²/day, for seven days every 28 days.

12. A kit of parts comprising the pharmaceutical composition according to claim 1 in a vial, cartridge or container and an injection device.

13. A method for preparing the pharmaceutical composition according to claim 1, comprising the steps of
(a) providing azacitidine; and
(b) mixing the azacitidine with dimethyl sulfoxide to provide the pharmaceutical composition.

14. The method of claim 13, further comprising the step of
(c) sterilization of the pharmaceutical composition by gamma ray irradiation and/or by filtration through a suitable membrane filter.

15. An infusion composition comprising the pharmaceutical composition of claim 1 and an infusion solution.

16. The infusion composition of claim 15 obtainable by mixing the pharmaceutical composition and an infusion solution.

17. The infusion composition of claim 16, wherein the infusion solution has a temperature of 0° C. to 15° C.

18. The infusion composition of claim 15, wherein the concentration of the azacitidine is 1 to 10 mg/mL.

19. The infusion composition of claim 15, wherein the concentration of the azacitidine is 1.5 to 4 mg/mL.

20. The infusion composition of claim 15 that is essentially free of impurities.
